(19) United States
(12) Patent Application Publication
(10) Pub. No.: US 2005/0245952 A1
(43) Pub. Date: Nov. 3, 2005

(54) Apparatus and method for dermal punch and follicular unit circumferential incision

(76) Inventor: Alan S. Feller, Woodmere, NY (US)

Correspondence Address: KENYON & KENYON, 1500 K STREET NW, SUITE 700, WASHINGTON, DC, 20005, US

(21) Appl. No.: 10/834,338

(22) Filed: Apr. 29, 2004

(51) Int. Cl. A61B 17/32

(52) U.S. Cl. 606/170

(57) ABSTRACT

Apparatus and method are presented for a dermal punch for follicular unit circumferential incision. The apparatus may comprise a cylinder having an interior lumen of at least a two diameters. A cylindrical blade having a continuous cutting-edge formed thereon is shown, the cutting-edge may be in a plane normal to the central axis and may have an apex oriented in a direction away from a distal end. In another embodiment, a second right circular cylinder may surround the first and may be slidably coupled thereto. The second right circular cylinder may include a plurality of elongate members oriented toward the proximal end of the first right circular cylinder. Each of the plurality of elongate members may have an cutting-edge in a plane substantially normal to a central axis and may have an apex oriented in a direction away from the distal end.

### BACKGROUND

1. Technical Field

Embodiments of the present invention generally relate to surgical instruments. More particularly, embodiments relate to surgical instruments known as dermal punches and to surgical instruments used to make circular incisions.

2. Discussion

The problem of human hair loss affects both men and women of all races. The vast majority of hair loss occurs in men. The reasons for hair loss are well known and are not described herein. Many methods of restoring hair have been used over the years, some with great success. A present method of hair restoration involves the surgical harvesting of human hair follicles from a donor area on a patient. The hair follicles are then surgically implanted into the balding, or bald area of the patient.

In the field of surgical hair transplants, and as used herein, the term “follicular unit” is used to refer to a group of from about one to five follicular organs (more than five are of course possible). It is noted that follicular organs tend to grow in proximity to each other and hence form natural clusters or “follicular units.”

FIG. 1 shows a schematic representation of a single human hair follicular organ 100 and is included for reference. The follicular organ 100 generally lies at an angle in the scalp. The follicular organ 100 is comprised of a follicular cell lining 102, at the bottom of which is contained a bulb 104 from which a hair shaft 106 extends. The follicular organ 100 protrudes from the scalp through an opening in the epidermis 108, which is the outer skin layer. The epidermis is approximately 0.5-1.0 mm in depth. The follicular organ 100 lies predominantly in the dermis 110, or dermal layer, which is a layer of about 4 mm in depth. The dermal layer includes collagen fibers, which may be described as threads or fibers. The collagen fibers provide some support for the follicular organ 100. Each follicular organ 100 has attached to it a small muscle known as the pilierector muscle 112. This muscle 112 attaches to the follicular organ 100 at a point usually somewhat near the mid-point of the follicular organ 100 and is coupled at the opposite end to a sub-dermal layer 114. The follicular organ 100 also extends into the sub-dermal layer 114. The sub-dermal layer is comprised of fat. The distance between the dermal/sub-dermal interface and the skull 116 is approximately 3 mm. Other layers of the scalp known to those of skill in the art are not mentioned herein and may be, at least in part, reprented by the dashed lines above the skull 116. Of course, all depths described above are for reference and may vary depending on, for example, the race, sex, and age of each human subject.

As stated above, a present method of hair restoration involves the surgical harvesting of human hair follicles from a donor area on a patient. A present method of harvesting involves the excision of a long strip of tissue (i.e., epidermis, dermis, and sub-dermal tissue) from a donor area on the patient. The long strip of tissue may have about 500 to 3,000 follicular units embedded therein. The donor area may typically be located at the back of the patient's head. The excision of the long strip of tissue, however, may leave a long scar, which is visible through the remaining hair. Removal of individual follicular units using conventional dermal punches has been attempted and has not met with success because, for example, the use of conventional punches inserted about the entire perceived circumference of a follicular unit, to a depth great enough to facilitate removal of a follicular unit, may lead to portions of the follicular unit being sheared or shaved-off by the conventional punch, thus injuring of killing the follicular unit. As used herein “shearing” may mean the ripping off of tissue due to, for example, friction. Attempts to solve the problem include the sliding of the punch only part way down, and pulling the partially freed follicular unit with fine forceps until all remaining supporting dermis gives way. This, however, leads to crushing or tearing the follicular unit, as well as to lengthy delays between extractions of follicular units. Furthermore, conventional punches having a central lumen of a single diameter tend to become clogged with tissue and coagulating blood, forcing the surgeon to substitute a new clean dermal punch or to clean the dermal punch already in use, thus again leading to lengthy delays between extractions of follicular units.

### BRIEF DESCRIPTION OF THE DRAWINGS

The various advantages of embodiments of the present invention will become apparent to one skilled in the art by reading the following specification and appended claims, and by referencing the following drawings, wherein like reference numerals identify like terms.

FIG. 1 shows a schematic representation of a single human hair follicular organ 100 and is included for reference.

FIG. 2A is an illustration of a device in accordance with an embodiment of the invention.

FIG. 2B is an illustration of a device in accordance with a second embodiment of the invention.

FIG. 2C is an illustration of a device in accordance with a third embodiment of the invention.

FIG. 3 is a cutaway view of a dermal punch in accordance with an embodiment of the invention.

FIG. 4A is an enlarged cutaway view of an portion of dermal punch 400 which includes both a cylindrical blade 402 and a cylindrical shaft 404 in accordance with an embodiment of the invention.

FIG. 4B is an enlarged cutaway view of a first alternate portion of dermal punch 400′, which includes both the cylindrical blade 402′ and the cylindrical shaft 404′ in accordance with an embodiment of the invention.

FIG. 4C is an enlarged cutaway view of a second alternate portion of dermal punch 400″ which includes both the cylindrical blade 402″ and the cylindrical shaft 404″ in accordance with an embodiment of the invention.

FIG. 5 is a cutaway view of a collar 500 (similar to 204, FIG. 2) in accordance with an embodiment of the invention.

FIG. 6 is a three dimensional view of collar 600 in accordance with the invention.

FIG. 7 illustrates an alternate embodiment of collar 700 including a plurality of sharpened elongate members 710 according to an embodiment of the invention.

FIGS. 8A and 8B illustrate alternate embodiments of a device 800 for follicular unit circumferential incisions in accordance with embodiments of the invention.

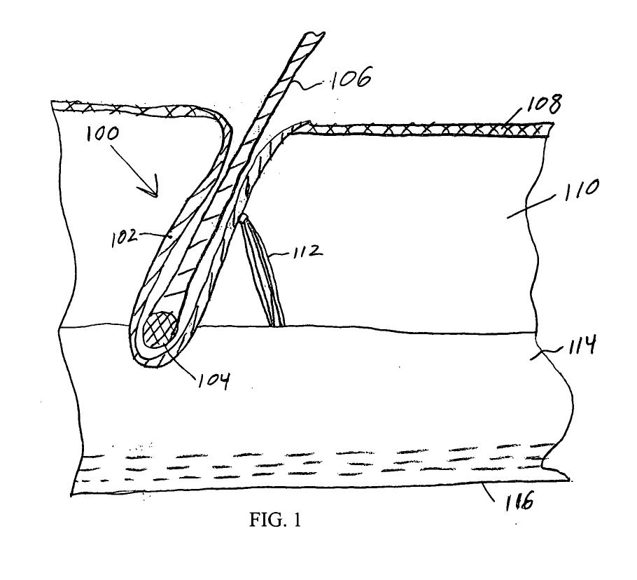

### DETAILED DESCRIPTION

The present apparatus seeks to overcome at least the above-described problems. In embodiments in accordance with the invention described herein, an improved dermal punch is described. The improved dermal punch includes at least a central lumen having multiple internal diameters to allow for less restricted movement of excised tissue within the central lumen (as compared to conventional punches having a single diameter central lumen). Also described is a collar having a plurality of elongated sharpened members extending therefrom. The collar may surround a dermal punch and the elongated sharpened members may be forced beyond the cutting-edge of the dermal punch, thus creating a plurality of deeper incisions (i.e., a circumferential partially scored and interrupted perforation pattern) about the perceived circumference of a follicular unit. The plurality of sharpened members cutting to a depth greater that the cutting-edge of the dermal punch may weaken the structural integrity of the surrounding dermal layer (for example, by severing collagen fibers and/or the pilierector muscle) and thus facilitate extraction of the follicular unit by reducing the amount of force needed to pull the follicular unit free of any remaining connective tissue. Furthermore, the plurality of sharpened members may reduce the chance of cutting an individual follicular organ in the targeted follicular unit, or shearing or shaving-off a follicular unit, by virtue of at least their being space between sharpened members and by perhaps allowing the follicular unit to move or flex as a result of the interrupted perforation pattern of the plurality of sharpened members.

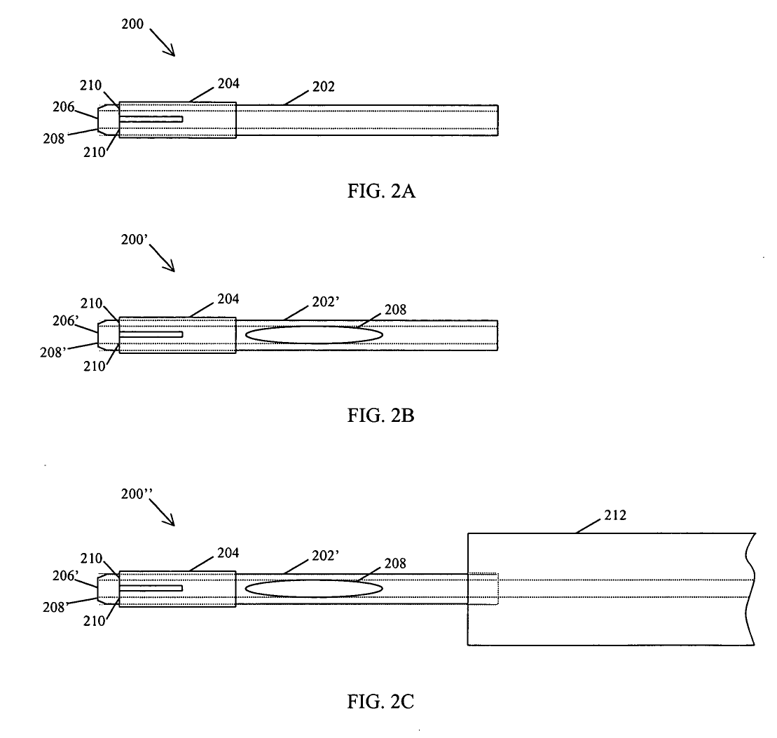

FIG. 2A is an illustration of a device 200 in accordance with an embodiment of the invention. The device 200 may comprise a dermal punch 202 and a collar 204 slidably coupled to a distal end thereto. The dermal punch 202 may include a central lumen 206 having multiple diameters (not shown) extending at least part way, but preferably completely through the dermal punch 202. The dermal punch may include a circular blade 208 formed into the distal end thereof. The dermal punch 202 may be used to, for example, isolate a follicular unit prior to the follicular unit's extraction from a human donor area and to penetrate surrounding tissue to a first depth, which may be about half the depth of the follicular unit. The collar 204 may include a plurality of elongated sharpened members 210 to penetrate surrounding tissue to a second depth, greater than the first to preferably sever additional surrounding tissue about a lower portion of the follicular unit.

FIG. 2B is an illustration of a device 200′ in accordance with a second embodiment of the invention. The device 200′ may comprise a dermal punch 202′ and a collar 204 slidably coupled to a distal end thereto. The dermal punch 202′ may include a central lumen 206′ having multiple diameters (not shown) extending at least part way, but preferably completely through the dermal punch 202′. The dermal punch 202′ may include a circular blade 208′ formed into the distal end thereof. The dermal punch 202′ may be used to, for example, isolate a follicular unit prior to the follicular unit's extraction from a human donor area and to penetrate surrounding tissue to a first depth, which may be about half the depth of the follicular unit. The collar 204 may include a plurality of elongated sharpened members 210 to penetrate surrounding tissue to a second depth, greater than the first, to preferably sever additional surrounding tissue about a lower portion of the follicular unit. The dermal punch 202′ may include an aperture or escape port 208, which serves, in one aspect to allow accumulated tissue in the central lumen 206′ to escape so that the central lumen 206′ does not become clogged with tissue. In another aspect, the port 208 serves as an air hole to allow air to escape from the central lumen 206′ when making incisions. It is noted that the central lumen 206 (if extended through the entire length of the dermal punch 202) and central lumen 206′ may also, or alternatively, serves as an air hole to allow air to escape from the central lumen 206, 206′ when making incisions. The port 208 may be provided at a location on the dermal punch 202′ that is sufficiently proximal from the blade 208′ and from the collar 204 in a non-extended position) so that the port 208 preferably remains above the skin when the blade 208′ is inserted into the scalp. Preferably, the port 208 is located about 4-12 mm or greater from the blade 208. The port 208 may be provided with a variety of geometries, but is preferably sufficiently large so that accumulated tissue can escape from the central lumen 206′. The port 208 may preferably be elliptical. In an exemplary configuration, a first axis of the ellipse may have a length that is about ninety percent of the inner diameter of the central lumen 206′ and a second axis may have a length that is about five times the length of the first axis or greater. Such a configuration is advantageous in allowing accumulated tissue to easily escape from the central lumen 206′ when forming numerous incisions in the scalp.

FIG. 2C is an illustration of a device 200″ in accordance with a third embodiment of the invention. The device 200″ may comprise a dermal punch 202, 202′ and a collar 204 slidably coupled to a distal end thereto. The dermal punch 202, 202′ may include a central lumen 206, 206′ having multiple diameters (not shown) extending at least part way, but preferably completely through the dermal punch 202, 202′. The dermal punch 202, 202′ may include a circular blade 208, 208′ formed into the distal end thereof. The dermal punch 202, 202′ may be used to, for example, isolate a follicular unit prior to the follicular unit's extraction from a human donor area and to penetrate surrounding tissue to a first depth, which may be about half the depth of the follicular unit. The collar 204 may include a plurality of elongated sharpened members 210 to penetrate surrounding tissue to a second depth, greater than the first, to preferably sever additional surrounding tissue about a lower portion of the follicular unit. The dermal punch 202, 202′ may include an aperture or escape port 208, which serves, in one aspect to allow accumulated tissue in the central lumen 206′ to escape so that the central lumen 206′ does not become clogged with tissue. In another aspect, the port 208 serves as an air hole to allow air to escape from the central lumen 206′ when making incisions. It is noted that the central lumen 206 (if extended through the entire length of the dermal punch 202) and central lumen 206′ may also, or alternatively, serves as an air hole to allow air to escape from the central lumen 206, 206′ when making incisions. The port 208 may be provided at a location on the dermal punch 202′ that is sufficiently proximal from the blade 208′ and for the collar in a non-extended position) so that the port 208 preferably remains above the skin when the blade 208′ is inserted into the scalp. Preferably, the port 208 is located about 4-12 mm or greater from the blade 208. The port 208 may be provided with a variety of geometries, but is preferably sufficiently large so that accumulated tissue can escape from the central lumen 206′. The port 208 will preferably be elliptical. In an exemplary configuration, a first axis of the ellipse will have a length that is about ninety percent of the inner diameter of the central lumen 206′ and a second axis will have a length that is about five times the length of the first axis or greater. Such a configuration is advantageous in allowing accumulated tissue to easily escape from the central lumen 206′ when forming numerous incisions in the scalp. The device 200″ further includes a handle 212, which may be fixedly or releasably secured to the dermal punch 202, 202′. The handle may include a handle central lumen 214, which may allow for an extension of the central lumen 206, 206′ of the dermal punch to facilitate, for example, the passage of air.

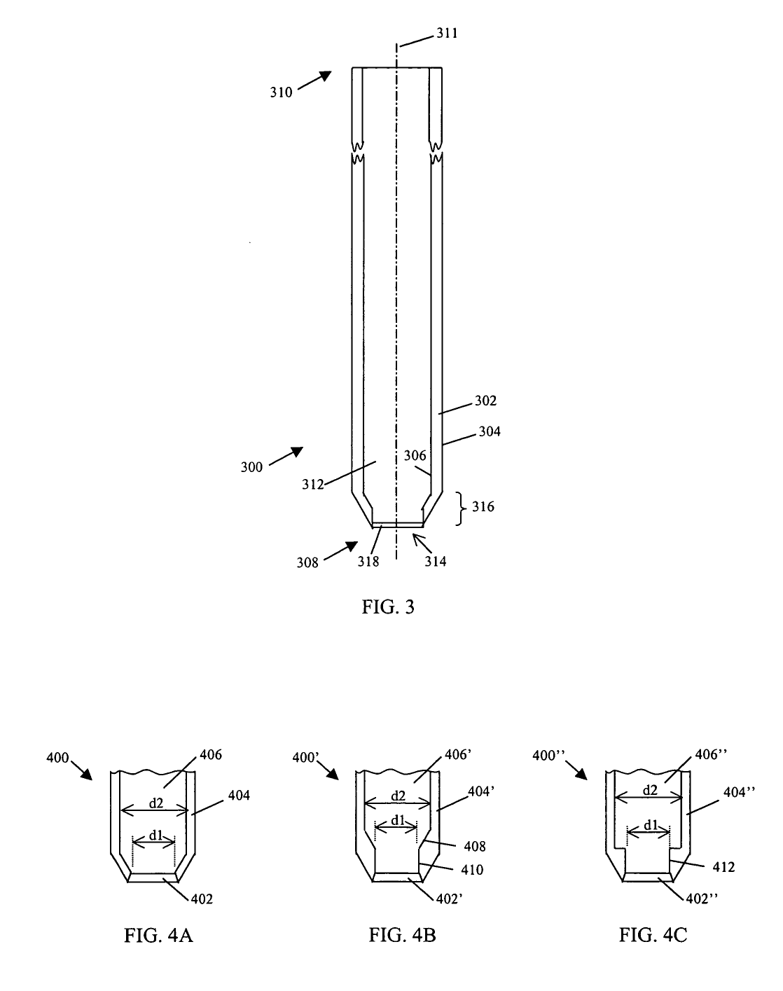

FIG. 3 is a cutaway view of a dermal punch 300 in accordance with an embodiment of the invention. In one embodiment the dermal punch 300 may comprise an elongate right circular cylindrical shaft 302 having an exterior wall 304, an interior wall 306, a distal end 308, and a proximal end 310. The distal end 308 and proximal end 310 are preferably normal to a central axis 311 extending between the proximal end 310 and the distal end 308. The elongate cylindrical shaft 302 may include a central lumen 312 substatially coaxially sharing the central axis 311 of the cylindrical shaft 302.

In one embodiment, the exterior wall 304 and the interior wall 306 are preferably substantially free of milling marks or other artifices of a manufacturing process. Both the exterior wall 304 and the interior wall 306 may preferably be machined, coated, plated, polished, or the like to provide a surface finish that will allow dermal tissue to slide alongside or pass through and/or alongside of the exterior wall 304 and the interior wall 306. The exterior wall 304 and the interior wall 306 may have the same surface finish, however, they may have different finishes without departing from the scope of the invention.

The cylindrical shaft 302 may have an outside diameter ranging from about 0.8 to 1.6 mm, and preferably from about 1.0 to 1.2 mm. In one embodiment, the cylindrical shaft 302 has an outside diameter of about 1.2 mm. The inside diameter of the cylindrical shaft 302, as formed by the central lumen 312, may range from about 0.6 to 1.2 mm, and preferably from about 0.9 to 1.1 mm. In one embodiment, the central lumen 312 has a diameter of about 1.0 mm. The inside diameter of the cylindrical shaft 302 is preferably wide enough to encircle at least one follicular unit. The follicular unit may comprise from about one to five human hair follicles, although more follicles in a follicular unit are possible. The minimum length of the cylindrical shaft 302 preferably accommodates insertion of the proximal end 308 through the epidermis and into the dermis and includes sufficient non-inserted length to allow a collar, similar to 204, FIGS. 2A, 2B, 2C, to be slidably positioned around the exterior surface of the cylindrical shaft 302, to remain external to the epidermis (i.e., above the external surface of the skin). In one embodiment, the distal end 308 of the cylindrical shaft may be inserted to a depth of about one quarter to three quarters of the depth of the follicular unit and preferably to a depth of about one-half the depth of the follicular unit. A follicular unit may have a depth, from epidermal surface to subdermal fatty layer, of about 5 mm. The depth of a follicular unit, as well as its perceived diameter, varies at least, however, from human to human and from racial group to racial group and as a function of age. In one embodiment, the length of the cylindrical shaft 302 may range from about 1.5 to 3 cm. In one embodiment, the length of the cylindrical shaft 302 is about 2 cm. If, in an embodiment, such as the embodiment of FIGS. 2B and 2C an exit port 208 is provided, then the minimum length of the cylindrical shaft is preferably increased to allow the collar 204 to be positioned below the exit port yet still remain external to the epidermis when the distal end 308 of the cylindrical shaft 302 has penetrated to a depth of about one-quarter to three-quarters of the depth of a follicular unit. The minimum length of the cylindrical shaft 302 also preferably includes the length of the cylindrical shaft that is fixedly or removably inserted into a handle, such as handle 212, FIG. 2C.

The dermal punch may further comprise a cylindrical blade 314. In one embodiment, the cylindrical blade 314 may have a truncated conical external cross-section 316. A knife-edge or cutting-edge 318 may be formed at distal end 308 of the cylindrical shaft 302. The cutting-edge 318 may comprise a substantially smooth and continuous (e.g., non-serrated) edge. The cutting-edge 318 may be formed such that the apex of the cutting-edge (i.e., that portion of the cylindrical blade 318 that first penetrates the epidermis) lies adjacent to the blade exterior wall 304, the interior wall 306, or disposed anywhere between. An ability to select apex location may allow greater success rates for follicular unit extraction as moving the apex to lie adjacent to the interior wall 306 allows for less friction as the tissue passes into the central lumen, when compared to a case where the apex is adjacent to the exterior wall 304. In a preferred embodiment, the apex is located adjacent to the interior wall 306. Those of skill in the art will understand how to form a cutting-edge on the distal end 308 of the cylindrical shaft 302.

The distal end 308 of the cylindrical shaft 302 may be formed at an angle to the central axis of the cylindrical shaft 302; for example, to facilitate reduction of distortion of tissue as the cylindrical blade penetrates the epidermal and dermal layers of the human skin. The angle between the exterior wall 304 at the distal end of the cylindrical shaft 302 and the central axis of the cylindrical shaft may range from about 10 to 60 degrees, and more preferably from about 20 to 40 degrees. In one embodiment, the angle is about 30 degrees.

In operation, a dermal punch, such as that disclosed herein, may penetrate the skin to create a substantially circular incision therein. As the dermal punch enters the skin (e.g., epidermis and dermis) tissue and living organ material including, for example, a follicular unit may preferably enter the central lumen. If the central lumen has the same diameter as the circular blade, the tissue will occupy the entirety of the width of the central lumen. Blood from the tissue and from the incision itself may seep into or be forced into the interstitial region between the outer edge of the tissue and the inner wall formed by the central lumen. Blood tends to coagulate when exposed to air and is sticky to the touch. Tissue, furthermore, exhibits a degree of friction against the interior wall of the lumen. At least these factors contribute to the tissue becoming lodged in the lumen, which often leads to toaget tissue torsion (twisting) and/or tearing; and contributes to delay in surgical procedures as the surgeon must pause from making circular incisions and either dislodge tissue and/or clean the dermal punch. In embodiments disclosed herein, the problem of lodged tissue is alleviated by the use of a central lumen 312 whose diameter is greater than the diameter of the cylindrical blade 318.

FIG. 4A is an enlarged cutaway view of an portion of dermal punch 400 which includes both a cylindrical blade 402 and a cylindrical shaft 404 in accordance with an embodiment of the invention. As illustrated in FIG. 4A, a central lumen 406 may have a diameter that varies along the length of the dermal punch 400. In one embodiment, the central lumen may have a first diameter, d1, about equal to the diameter of the cylindrical blade 402 at the distal end of a dermal punch. In the embodiment, the central lumen diameter may increase in size from the first diameter to a second diameter, d2, greater than the first. The second diameter, d2, may be approximately 1.1 to 2 times as large as the first diameter, d1. The area in which the increase in diameter may occur is an area immediately adjacent to the cylindrical blade and extending along the length of the central lumen. The angle at which the diameter increase occurs may be varied. In one embodiment, as shown in FIG. 3A, the area in which the increase in diameter may occur comprises an interior wall surface having an angle approximately equal to the angle of the exterior wall surface.

FIG. 4B is an enlarged cutaway view of a first alternate portion of dermal punch 400′, which includes both the cylindrical blade 402′ and the cylindrical shaft 404′ in accordance with an embodiment of the invention. As illustrated in FIG. 4B, the interior wall of the cylindrical shaft 404′ may include a right circular conical surface 408 having a first diameter d1 about equal to the diameter of the cylindrical blade 402′, and a second diameter d2, larger than the first and further separated from the cylindrical blade 402′. In one embodiment, the length of the right circular conical surface 408 may range from about one-quarter to three-quarters of the diameter of the circular blade 402′. The interior wall of the cylindrical shaft 404′ may further comprise a right circular cylinder 410 having a fixed diameter about equal to about d1. The right circular cylinder extends from the narrowest diameter of the right circular conical surface 408 and to the cylindrical blade 402′. In one embodiment, the length of the right circular cylinder 410 may range from about one quarter to three-quarters of the diameter of the circular blade. It is noted that those of skill in the art may refer to the feature described in the illustration of FIG. 4B as a countersink.

FIG. 4C is an enlarged cutaway view of a second alternate portion of dermal punch 400″ which includes both the cylindrical blade 402″ and the cylindrical shaft 404″ in accordance with an embodiment of the invention. In one embodiment, the interior wall of the cylindrical shaft 404″ may include a right circular cylindrical surface 412 having a first diameter d1 about equal to the diameter of the cylindrical blade 402″, and a second diameter d2, larger than the first and further separated from the cylindrical blade 402″. In one embodiment, the length of the right circular cylindrical surface 412 may range from about one-quarter to three-quarters of the diameter of the circular blade 402″. It is noted that those of skill in the art may refer to the feature described in the illustration of FIG. 4B as a counterbore.

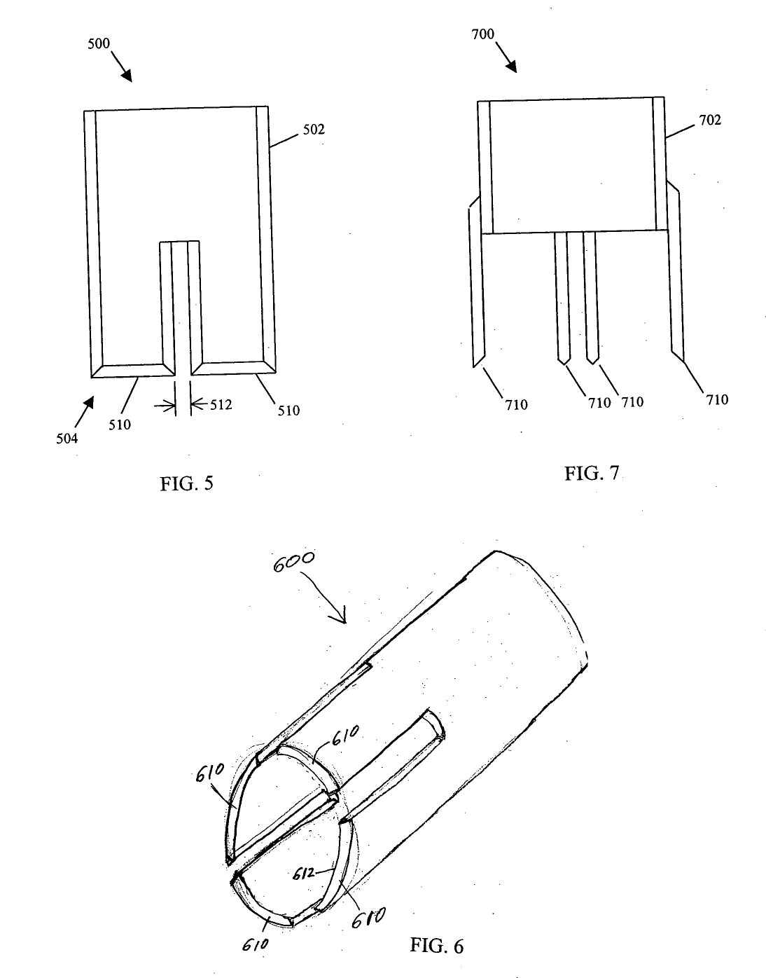

FIG. 5 is a cutaway view of a collar 500 (similar to 204, FIG. 2) in accordance with an embodiment of the invention. The collar 500 comprises a hollow right circular cylinder 502 having a distal end 504 and a proximal end 506. The collar 500 further comprises a plurality of sharpened elongate members 510. It is noted that a sharpened elongate member 510 may be connected to the hollow right circular cylinder 502 by virtue of both the elongate member 510 and the hollow right circular cylinder 502 having been machined from the same piece of material (e.g., the elongate member 510 and the hollow right circular cylinder 502 are one integral unit). It is also noted that if the plurality of elongate members 510 are not one integral unit or if they are an integral unit that is separate from the cylinder, then each of the plurality of elongate members 510, or groups of them, may be connected to the hollow circular cylinder 502 by welding, soldering, brazing, epoxying, gluing, or otherwise bonding or friction fitting each elongate member 510, or the entire plurality of them 510 to the right circular cylinder 502. The preceding list is meant to be exemplary and is not limiting.

FIG. 6 is a three dimensional view of collar 600 in accordance with the invention. As illustrated in FIG. 6, in one embodiment, a sharpened elongate member 610 may comprise a three dimensional rectangular beam whose profile is curved to match that of the right circular cylinder 602. The distal end of each elongate member 610 (or the plurality of elongate members 610) may be sharpened to a cutting-edge 612. The cutting-edge 612 may comprise a substantially smooth and continuous (e.g., non-serrated) edge. The cutting-edge 612 may be formed such that the apex of the cutting-edge lies adjacent to the interior of the right circular cylinder 602, the exterior of the right circular cylinder 602, or may be disposed anywhere between. In the embodiment as illustrated in FIG. 6, the apex of the cutting-edge 612 lies adjacent to the interior of the right circular cylinder 602. Those of skill in the art will understand how to form a cutting-edge on the edges of the elongate members 610.

FIG. 7 illustrates an alternate embodiment of collar 700 including a plurality of sharpened elongate members 710 according to an embodiment of the invention. In the embodiment of FIG. 7, the plurality of elongate members 710 are comprised of small diameter spikes connected to a right circular hollow cylinder 702. The connection may be by welding, soldering, brazing, epoxying, gluing, or some other attachment mechanism as known to those of skill in the art. The preceding list is meant to be exemplary and is not limiting. The point of connection may be flattened or otherwise smoothed to avoid discontinuities in surface geometry that may tear tissue or snag the slidably removable collar 700 as it exits an incision.

The length of the collar 500, 600, 700 may range from about 1 to about 2 cm, and preferably from about 0.8 cm to 1.60 cm. In one embodiment, the length of the collar 500, 600, 700 is about 1.6 cm. The length of each of the plurality of elongate members 510, 610, 710 may range from 0.25 to 0.75 cm. In one embodiment, the length of the plurality of elongate members is about 0.4 cm. The distance between the plurality of sharpened elongate members (e.g., the distance 512 as between members 510 in FIG. 5) may vary depending on race, sex, age, and health of a follicular unit donor. In one embodiment, the distance 512 is about 1 mm.

The number of elongate members 510, 610, 710 may range from about three to eight. In one embodiment, the number of elongate members is four. In one embodiment, the elongate members are formed, for example, from the right circular cylinder by forming slots, for example by cutting, milling, or grinding the slots into a metallic tube, from the rim of the metallic tube to a predetermined distance along the body of the metallic tube, thereby segmenting the one end of the metallic tube into a plurality of elongate members. The rim may have been previously sharpened to form a single cutting-edge; segmenting the rim accordingly provides a plurality of individual cutting-edges. Other methods of machining the collar, including the hollow right circular cylinder and the plurality of elongate members will be know to those of skill in the art.

The dermal punch and collar, such as (202, 204 of FIG. 2) may be made of surgical steel, carbon steel, or any other metal or ceramic capable of being machined to the suggested geometries and further capable holding a sharpened cutting-edge for at least a limited number of incisions into human skin. The limited number of incisions may range from at least 25 to 500, and preferably may range from at least 200 to 500. In one embodiment, the cutting-edge(s) on the dermal punch (e.g., 202, 202′, FIG. 2) and collar (e.g., 204, 204′, FIG. 2) may be resharpened. In one embodiment, the dermal punch (e.g., 202, 202′, FIG. 2) and collar (e.g., 204, 204′, FIG. 2) may be disposable.

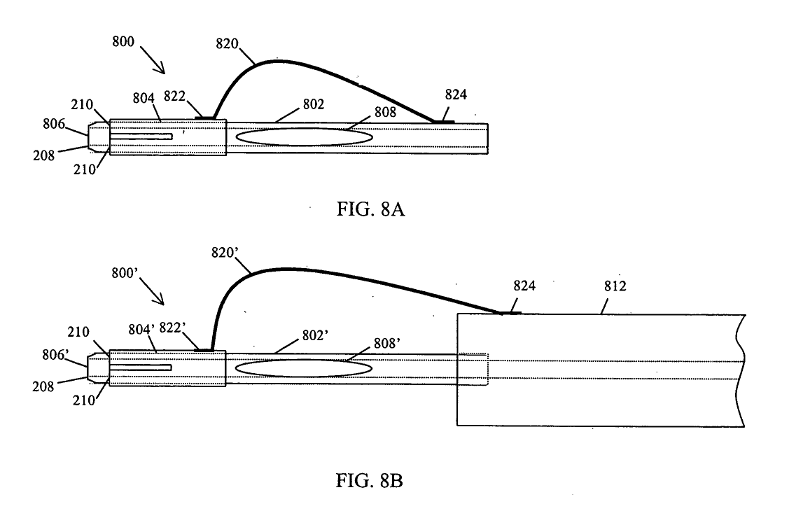

FIGS. 8A and 8B illustrate alternate embodiments of a device 800, 800′ for follicular unit circumferential incisions in accordance with embodiments of the invention. As illustrated in FIG. 8A, the device 800 may be comprised of a dermal punch 802, a collar 804 surrounding an exterior surface of the dermal punch 802 and slidably coupled thereto and coaxial therewith, and a flexible member 820 having a mechanical memory. The device 800 may further include a central lumen 806 and an exit port 808. The flexible member 806 may include a first end 822 mechanically coupled to the collar 804 and a second end 824 mechanically coupled to the dermal punch 802.

As illustrated in FIG. 8B, the device 800′ may be comprised of a dermal punch 802′, a collar 804′ surrounding an exterior surface of the dermal punch and slidably coupled thereto and coaxial therewith, a flexible member 820′ having a mechanical memory, and a handle 812. The flexible member 820′ may include a first end 822′ mechanically coupled to the collar 804′ and a second end 824′ mechanically coupled, via the handle 812, to the dermal punch 802′. The device 800′ may further include a central lumen 806′ and an exit port 808′.

The flexible member 806, 806′ may be any flexible material that may deform from a first shape by application of physical force and return substantially to the first shape after removal of the physical force. In one embodiment, the flexible member 806, 806′ may be a ribbon of spring steel. In the illustrated embodiment of FIGS. 8A and 8B, at least one flexible member 806, 806′ is shown, however, those of skill in the art will understand that more than one flexible member 806, 806′ may be included without departing from the scope of the invention. Furthermore, those of skill in the art will understand the a plurality of flexible members (similar to 806, 806′) may be included and may be mechanically coupled to the collar 804, 804′ and/or the dermal punch 802, 802′ either at regular or irregular intervals about the circumferences thereof.

Mechanical coupling of the flexible member 806, 806′ to the collar 804, 804′ and/or to the dermal punch 802, 802′ may be, for example, by welding, soldering, brazing, epoxying, gluing or some other attachment mechanism as know to those of skill in the art. The preceding list is meant to be exemplary and is not limiting. Mechanical coupling of the flexible member 806, 806′ to the collar 804, 804′ and/or to the dermal punch 802, 802′ may be indirect. For example, as illustrated in the embodiment of FIG. 8B, mechanical coupling of the flexible member 806′ to the dermal punch 802′ may be made by physical attachment of the second end 812′ of the flexible member 806′ to a handle 808 in which the dermal punch 802′ is fixedly or removably attached. Furthermore, it is noted that the mechanism of attachment may be dictated by the choice of materials used for the dermal punch 802, 802′, the collar 804, 804′, the flexible member 806, 806′, and the handle 808.

In one embodiment, actuation of the flexible member 806, 806′ to drive and to retract the collar 804, 804′ along the length of the dermal punch 802, 802′ may be through application of a physical force to the flexible member 806, 806′ in a direction normal to a central axis of the dermal punch 802, 802′.

Those of skill in the art will understand that the linear length of the flexible member 806, 806′ is preferably chosen such that when no physical force is applied to the flexible member 806, 806′, the collar 804, 804′ is maintained at approximately four to six mm from the cutting-edge of the circular blade 208; and when some amount of physical force is applied to the flexible member 806, 806′, the collar 804, 804′ is maintained at a approximately two to four mm beyond the cutting-edge of the circular blade 208.

In operation, a method of using an embodiment of the invention disclosed herein may include: anesthetizing a follicular unit donor area of a subject; shaving the donor area to remove excess length of individual ones of shafts of hair; aligning a device for follicular unit circumferential incisions (e.g., 200, 200′, 200″, 800, 800′, 900) to be substantially parallel with a perceived axis of the follicular unit; inserting the dermal punch having a cylindrical blade at is distal end through the epidermis and into the dermis to a depth of about one-half the perceived depth of the follicular unit; driving forward the collar having sharpened elongated members at its distal end such that the most distal ends of the sharpened elongated members penetrate the dermis beyond the most distal end of the circular blade of the dermal punch; and retracting the collar and the dermal punch from the incision. The method may further include gently pulling on the now partially scored and intermittently perforated tissue circumferentially surrounding the follicular unit until any remaining supportive tissue tears and the follicular unit is free.

It is noted that penetration of epidermis may require slight rotation of the cylindrical blade (e.g., 208, FIG. 2). The rotation may be back-and-forth on the order of tens of degrees and preferably less than 10 degrees, if the device in accordance with the invention is manipulated by hand. After penetration of the circular blade (e.g., 208, FIG. 2) of the dermal punch (e.g., 202, FIG. 2) to a desired depth is accomplished, it is preferable that no substantial further rotation of the dermal punch occur and that a penetrating incision may be made coaxial to the dermal punch by the plurality of sharpened elongated members (e.g., 210, FIG. 2).

Those skilled in the art can appreciate from the foregoing description that the broad techniques of the embodiments of the present invention can be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

1. An apparatus comprising:
an elongate right circular cylinder having:
an interior wall having at least a first diameter and a second diameter, the second diameter being greater than the first diameter, the interior wall forming a central lumen substantially coaxial to a central axis of the right circular cylinder,
an exterior wall having a third diameter, the third diameter being greater than the second diameter,
a distal end, and
a proximal end; and
a cylindrical blade adjacent to the proximal end and including a blade central lumen, the blade central lumen concentric with the central axis, the cylindrical blade having a continuous cutting-edge formed thereon, the cutting-edge in a plane normal to the central axis and having an apex oriented in a direction away from the distal end, wherein the first diameter is substantially equal to a diameter of the continuous cutting-edge.

2. The apparatus of claim 1, wherein the cylindrical blade has a truncated conical external cross-section.

3. The apparatus of claim 1, wherein the apex of the cutting-edge is disposed adjacent to the exterior wall.

4. The apparatus of claim 1, wherein the apex of the cutting-edge is disposed adjacent to the interior wall.

5. The apparatus of claim 1, wherein the apex of the cutting-edge is disposed substantially midway between the exterior wall and the interior wall.

6. The apparatus of claim 1, wherein the right circular cylinder and the cylindrical blade are formed from a single piece of stock material.

7. The apparatus of claim 1, wherein the central lumen increases in diameter along the central axis from the proximal end to the distal end.

8. The apparatus of claim 1, wherein a change from the first diameter to the second diameter is an abrupt change.

9. The apparatus of claim 1, wherein a change from the first diameter to the second diameter is a gradual change.

10. The apparatus of claim 1, wherein the diameter of the central lumen at the proximal end is less than the diameter of the central lumen at the distal end.

11. The apparatus of claim 1, wherein a change from the first diameter to the second diameter is realized as a counterbore.

12. The apparatus of claim 1, wherein a change from the first diameter to the second diameter is realized as a countersink.

13. The apparatus of claim 1, further comprising an aperture penetrating the right circular cylinder from the external wall into the central lumen.

14. The apparatus of claim 13, wherein the aperture is elliptical in shape.

15. The apparatus of claim 1, further comprising a handle to one of fixedly and removably couple to the distal end of the right circular cylinder.

16. The apparatus of claim 15, wherein the handle includes a first lumen, coaxial to the central lumen of the right circular cylinder and mechanically coupled thereto.

17. An apparatus comprising:
a first elongate right circular cylinder having:
an interior wall having at least a first diameter and a second diameter, the second diameter being greater than the first diameter, the interior wall forming a central lumen substantially coaxial to a central axis of the first elongate right circular cylinder,
an exterior wall having a third diameter, the third diameter being greater than the second diameter,
a distal end, and
a proximal end;
a cylindrical blade adjacent to the proximal end and including a blade central lumen, the blade central lumen concentric with the central axis, the cylindrical blade having a continuous cutting-edge formed thereon, the cutting-edge in a plane normal to the central axis and having an apex oriented in a direction away from the distal end, wherein the first diameter is substantially equal to a diameter of the continuous cutting-edge; and
a second elongate right circular cylinder surrounding the first elongate right circular cylinder and slidably coupled thereto, the second elongate right circular cylinder including a plurality of elongate members oriented toward the proximal end of the first right circular cylinder, each of the plurality of elongate members having an elongate member cutting-edge in a plane substantially normal to the central axis and having an elongate member cutting-edge apex oriented in a direction away from the distal end.

18. The apparatus of claim 17, wherein the cylindrical blade has a truncated conical external cross-section.

19. The apparatus of claim 17, wherein the apex of the cutting-edge is disposed adjacent to the exterior wall.

20. The apparatus of claim 17, wherein the apex of the cutting-edge is disposed adjacent to the interior wall.

21. The apparatus of claim 17, wherein the apex of the cutting-edge is disposed substantially midway between the exterior wall and the interior wall.

22. The apparatus of claim 17, wherein the right circular cylinder and the cylindrical blade are formed from a single piece of stock material.

23. The apparatus of claim 17, wherein the central lumen increases in diameter along the central axis from the proximal end to the distal end.

24. The apparatus of claim 17, wherein a change from the first diameter to the second diameter is an abrupt change.

25. The apparatus of claim 17, wherein a change from the first diameter to the second diameter is a gradual change.

26. The apparatus of claim 17, wherein the diameter of the central lumen at the proximal end is less than the diameter of the central lumen at the distal end.

27. The apparatus of claim 17, wherein a change from the first diameter to the second diameter is realized as a counterbore.

28. The apparatus of claim 17, wherein a change from the first diameter to the second diameter is realized as a countersink.

29. The apparatus of claim 17, further comprising an aperture penetrating the right circular cylinder from the external wall to the central lumen.

30. The apparatus of claim 29, wherein the aperture is elliptical in shape.

31. The apparatus of claim 17, further comprising a handle to one of fixedly and removably couple to the distal end of the first right circular cylinder.

32. The apparatus of claim 31, wherein the handle includes a first lumen, coaxial to the central lumen of the right circular cylinder and mechanically coupled thereto.

33. The apparatus of claim 17, wherein each of the plurality of elongate members comprises a three dimensional rectangular beam having a curved profile to match a profile of the second right circular cylinder.

34. The apparatus of claim 17, wherein the elongate member cutting-edge comprises a substantially smooth and continuous edge.

35. The apparatus of claim 17, wherein the elongate member cutting-edge apex is disposed adjacent to an inner surface of the second right circular cylinder.

36. The apparatus of claim 17, wherein the elongate member cutting-edge apex is disposed adjacent to an outer surface of the second right circular cylinder.

37. The apparatus of claim 17, wherein the elongate member cutting-edge apex is disposed substantially midway between the inner surface and the outer surface of the second right circular cylinder.

38. The apparatus of claim 17, wherein the second right circular cylinder including the plurality of elongate members is formed from a single piece of stock material.

39. The apparatus of claim 17, wherein each of the plurality of elongate members comprises a small diameter spike.

40. The apparatus of claim 39, wherein the plurality of elongate members is connected to the second right circular cylinder by one of welding, soldering, brazing, epoxying, and gluing.

41. The apparatus of claim 17, further comprising, a flexible member having a mechanical memory, the flexible member including a first end mechanically coupled to the second right circular cylinder and a second end mechanically coupled to the first right circular cylinder.

42. The apparatus of claim 41, wherein the flexible member having a mechanical memory is a material that deforms from a first shape by application of a physical force and returns substantially to the first shape after removal of the physical force.

43. The apparatus of claim 31, further comprising a flexible member having a mechanical memory, the flexible member including a first end mechanically coupled to the second right circular cylinder and a second end mechanically coupled to the handle.

44. The apparatus of claim 43, wherein the flexible member having a mechanical memory is a material that deforms from a first shape by application of a physical force and returns substantially to the first shape after removal of the physical force.
